(19) United States
(12) Patent Application Publication
(10) Pub. No.: US 2026/0207456 A1
(43) Pub. Date: Jul. 23, 2026

(54) COLOR STABILIZATION OF FORMULAE WITH AN INDOLE

(71) Applicant: Conopco, Inc., d/b/a UNILEVER, Hoboken, NJ (US)

(72) Inventor: Jose Guillermo Rosa, Cheshire, CT (US)

(73) Assignee: Conopco, Inc., d/b/a UNILEVER, Hoboken, NJ (US)

(21) Appl. No.: 19/138,926

(22) PCT Filed: Nov. 28, 2023
(86) PCT No.: PCT/EP2023/083274

§ 371 Date: Jun. 13, 2025

(30) Foreign Application Priority Data
Dec. 21, 2022 (EP) 22215527.7

(51) Int. Cl. A61K 8/49; A61K 8/34; A61K 8/67; A61Q 19/00

(52) U.S. Cl. CPC A61K 8/492; A61K 8/347; A61K 8/675; A61Q 19/00; A61K 2800/10; A61K 2800/52

(57) ABSTRACT

The invention is directed to a color stable cosmetic composition comprising an indole of formula (I), and/or salts or derivatives thereof, and a substituted resorcinol.

### FIELD OF THE INVENTION

The present invention is directed to a color stable cosmetic composition with an indole and at least one substituted resorcinol.

### BACKGROUND OF THE INVENTION

The present invention relates to a color stable cosmetic composition and a method of making and using the same, such that the cosmetic composition has an indole and at least one substituted resorcinol.

Resorcinol (1,3-benzenediol) derivatives have been used to provide cosmetic benefits to skin and hair. 4-substituted resorcinol derivatives and their preparation have been used for skin lightening and other cosmetic uses; see, for example, U.S. Pat. Nos. 4,959,393, 6,132,740, U.S. Patent Application Publication No. 2008/0131382, and Japanese Published Patent Application Nos. JP 2001-010925 and JP 2000-327557. Resorcinol-type skin lightening agents, which can be synthesized using coumarin as starting material, are disclosed in U.S. Patent Application Publication No. 2004/0042983. However, some of these compounds can be difficult to formulate, as they are unstable in a cosmetically acceptable vehicle.

Oxidative instability is an undesirable characteristic of substituted resorcinols. In particular, if a substituted resorcinol oxidizes in a product, especially in a consumer product such a cosmetic composition, the product may turn into an undesirable color (e.g., discolor), and the consumer may avoid using the product. There is a need, therefore, for an agent that will stabilize substituted resorcinols against oxidation. In particular, there is a need for an agent that will prevent the oxidation of substituted resorcinols, especially in cosmetic compositions.

It would be desirable to have cosmetic compositions that contain resorcinol derivatives that are stabilized against factors such as discoloration, oxidation, general storage stability. The color stable cosmetic compositions comprising substituted resorcinols stabilized with an indole as described herein, fill this need.

The inventive compositions are aesthetically pleasing and have improved storage/oxidative stability.

### SUMMARY OF THE INVENTION

In a first aspect, a color stable cosmetic composition comprises: (a) a compound of formula (I):

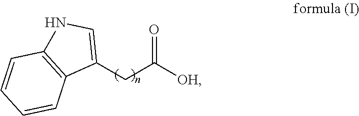

and/or salts or derivatives thereof; and (b) a substituted resorcinol, wherein n is 1 to 3; and wherein a weight ratio of the compound of formula (I) and the substituted resorcinol is about 1:40 to 40:1, or about 1:10 to 10:1, or about 5:1 to 1:5, or about 3:1 to 1:3.

In a second aspect, a color stable cosmetic composition comprises: (a) a compound of formula (I) as described herein, and/or salts or derivatives thereof; and (b) a substituted resorcinol; wherein an amount of the substituted resorcinol, by weight of the composition is about 0.01%-10%, or about 0.01%-5%, or about 0.01%-3%, or about 0.1%-5%, or about 0.1%-3%, or about 0.1%-1%; and wherein an amount of the compound of formula (I), by weight of the composition is about 0.01%-10%, or about 0.01%-5%, or about 0.1%-5%, or about 0.1%-3%, or about 0.1%-1%.

In a third aspect, a method is provided for making a color stable cosmetic composition comprising: mixing a compound of formula (I) as described herein, and/or salts or derivatives thereof, and (b) a substituted resorcinol; and wherein a weight ratio of the compound of formula (I) and the substituted resorcinol is about 1:40 or 40:1, or about 1:10 to 10:1, or about 5:1 to 1:5, or about 3:1 to 1:3, wherein the cosmetic composition is color stable for at least 10 days.

In a fourth aspect, a method is provided for making a color stable cosmetic composition comprising: mixing a compound of formula (I) as described herein, and/or salts or derivatives thereof, and (b) a substituted resorcinol; wherein an amount of the substituted resorcinol, by weight of the composition is about 0.01%-10%, or about 0.01%-5%, or about 0.01%-3%, or about 0.1%-5%, or about 0.1%-3%, or about 0.1%-1%; and wherein an amount of the compound of formula (I), by weight of the composition is about 0.01%-10%, or about 0.01%-5%, or about 0.1%-5%, or about 0.1%-3%, or about 0.1%-1%, wherein the cosmetic composition is color stable for at least 10 days.

In a fifth aspect, a method is provided for imparting a skin care benefit to skin in need of such treatment comprising: applying a color stable cosmetic composition to the skin, wherein the color stable cosmetic composition comprises a compound of formula (I) as described herein, and/or salts or derivatives thereof, and (b) a substituted resorcinol, and wherein a weight ratio of the compound of formula (I) and the substituted resorcinol is about 1:40 to 40:1, or about 1:10 to 10:1, or about 5:1 to 1:5, or about 3:1 to 1:3.

In a sixth aspect, a method is provided for imparting a skin care benefit to skin in need of such treatment comprising: applying a color stable cosmetic composition to the skin, wherein the color stable cosmetic composition comprises a compound of formula (I) as described herein, and/or salts or derivatives thereof, and (b) a substituted resorcinol, wherein an amount of the substituted resorcinol, by weight of the composition is about 0.01%-10%, or about 0.01%-5%, or about 0.01%-3%, or about 0.1%-5%, or about 0.1%-3%, or about 0.1%-1%; and wherein an amount of the compound of formula (I), by weight of the composition is about 0.01%-10%, or about 0.01%-5%, or about 0.1%-5%, or about 0.1%-3%, or about 0.1%-1%.

In yet another aspect, the invention also provides the use of a compound according to formula (I), as defined herein, or salts or derivatives thereof, and (b) a substituted resorcinol, and for the manufacture of, color stable cosmetic compositions for at least one skin benefit.

All other aspects of the present invention will more readily become apparent from the description and examples which follow.

### DETAILED DESCRIPTION OF THE INVENTION

Except in the examples, or where otherwise explicitly indicated, all numbers in this description indicating amounts of material or conditions of reaction, physical properties of materials and/or use may optionally be understood as modified by the word “about”.

All amounts are by weight of any of the compositions or components thereof, unless otherwise specified. It should be noted that in specifying any ranges of values, any particular upper value can be associated with any particular lower value.

For the avoidance of doubt, the term “comprising” is meant not to be limiting to any stated elements but rather to encompass non-specified elements of major or minor functional importance. Therefore, the listed steps, elements or options need not be exhaustive. Whenever the words “including” or “having” are used, these terms are meant to be equivalent to “comprising” as defined above.

The disclosure, as found herein, is to be considered to cover all embodiments as found in the claims as being multiply dependent upon each other irrespective of the fact that claims may be found without multiple dependency or redundancy.

Skin, as used herein, includes skin on the face, neck, chest, back, arms (including underarms), axilla, buttocks, hands, legs and scalp.

Skin care benefit, as used herein, refers to an improvement in a facial or body characteristic after topical application, for example, even skin tone such as skin brightening and/or glow, skin texture such as reduction in wrinkles, fine lines and/or age spots, dry skin, anti-acne or the like.

In a first aspect, a color stable cosmetic composition comprises compound of formula (I):

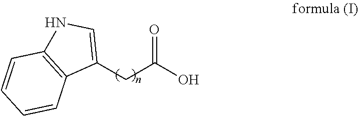

and/or salts or derivatives thereof; and a substituted resorcinol, wherein n is 1 to 3.

### Indole Compounds

The compound of formula (I) is an indole.

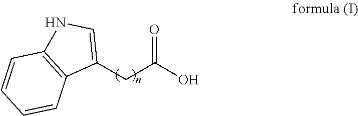

The indoles of formula (I) are indoles comprising a carboxylic acid. In one aspect, n is 1 to 3. The color stable cosmetic compositions as described herein, have a compound of formula (I), as shown below, and/or any salts or derivatives thereof. In one aspect, the cosmetic compositions have one or more indoles of formula (I) described herein, and/or salts or derivatives thereof.

Derivative(s), as used herein, refers to functional group substitutions on the compound. In one aspect, a derivative of formula (I) may be an ester derivative, such as an ethyl ester, or isopropyl ester. In one aspect, salts of the compound of formula (I) may be an alkali metal salt, an alkaline earth metal salt and/or an ammonium salts. In one aspect, an alkali metal salt may be, for example, a sodium (Na⁺), lithium (Li⁺), and/or potassium (K⁺) salt, an alkaline earth metal salt may be, for example, a calcium (Ca²⁺) or magnesium (Mg²⁺) salt, and an ammonium salt may be NH₄⁺ and/or NR₄⁺, with R being an alkyl group. Alkyl, as used herein, means carbon chains which may be linear or branched, or combinations thereof, containing the indicated number of carbon atoms. In particular embodiments, linear alkyl groups have 1-20 carbon atoms and branched alkyl groups have 3-20 carbon atoms. Examples of alkyl groups include methyl, ethyl, propyl, isopropyl, butyl, sec- and tert-butyl, pentyl, hexyl, heptyl, octyl, nonyl and the like.

In one aspect, the compound of formula (I) is 2-(1H-indol-3-yl)acetic acid (or “I3AA”), 3-(1H-indol-3-yl)propanoic acid (or “I3PA”), 4-(1H-indol-3-yl)butanoic acid (or “I3BA”), and/or mixtures and/or combinations thereof.

In one aspect, the amount of the compound of formula (I) as described herein, and/or any salt or derivative thereof included in a cosmetic composition is about 0.01% to about 10%, or about 0.01% to about 5%, or about 0.01% to about 3%, or about 0.01% to about 2%, or about 0.01% to about 1%, or about 0.1% to about 10%, or about 0.1% to about 5%, or about 0.1% to about 3%, or about 0.1% to about 2%, or about 0.1% to about 1%, or about 0.5% to about 10%, or about 0.5% to about 5%, or about 0.5% to about 3%, or about 1% to about 10%, or about 1% to about 5%, or about 1% to about 3%, by weight of the composition, including all ranges subsumed therein.

### Resorcinol Compounds

A resorcinol is a monohydroxy phenol compound (i.e., 1,3-dihydroxybenzene or 1,3-benzenediol) characterized by alcohol groups (—OH) at positions 1 and 3. The chemical structure of resorcinols may be modified, resulting in substituted resorcinols characterized by at least one substituent in the 2, 4, 5 or 6 position. Substituted, as used herein, refers to a compound in which one or more hydrogen atoms are replaced by another atom or group, e.g., substituent. In one aspect, the substituted resorcinol comprises at least one substituent comprising 5 to 11 carbon atoms, or 5 to 9 carbon atoms. In one aspect, the substituted resorcinol has at least one substituent comprised of an alkyl group or unsaturated alkyl group.

In one aspect, the substituted resorcinol is only substituted at position 4 with an alkyl group, e.g., the substituted resorcinol is a 4-substituted alkyl resorcinol, including but not limited to 4-methyl resorcinol, 4-ethyl resorcinol, 4-hexyl resorcinol, 4-cyclohexyl resorcinol, 4-propyl resorcinol, 4-isopropyl resorcinol, 4-butyl resorcinol, 4-pentyl resorcinol, 4-phenylethyl resorcinol, 4-cyclopentyl resorcinol, 4-cyclohexyl resorcinol, 4-heptyl resorcinol, 4-cycloheptyl resorcinol, 4-octyl resorcinol, 4-cyclooctyl resorcinol, 4-nonyl resorcinol, 4-decyl resorcinol, 4-undecyl resorcinol, 4-dodecyl resorcinol and/or combinations or mixtures thereof. Preferably, the substituted resorcinol is 4-ethylresorcinol, 4-hexylresorcinol, and 4-isopropylresorcinol. In one aspect, the substituted resorcinol is substituted at position 4 with a substituted or unsubstituted 5- or 6-membered ring carbon-based heterocyclic ring containing one or more heteroatoms selected from N, S or O, preferably a substituted 5-membered ring carbon-based heterocyclic ring containing two heteroatoms selected from N or S, e.g., isobutylamido thiazolyl resorcinol or thiamidol.

In one aspect, the amount of substituted resorcinol included in a cosmetic composition is about is about 0.01% to about 10%, or about 0.01% to about 5%, or about 0.01% to about 3%, or about 0.01% to about 2%, or about 0.01% to about 1%, or about 0.01% to about 0.75%, or about 0.01% to about 0.5%, or about 0.1% to about 10%, or about 0.1% to about 5%, or about 0.1% to about 3%, or about 0.1% to about 2%, or about 0.1% to about 1%, or about 0.1% to about 0.75%, or about 0.1% to about 0.5%, or about 0.5% to about 10%, or about 0.5% to about 5%, or about 0.5% to about 3%, or about 1% to about 10%, or about 1% to about 5%, or about 1% to about 3%, by weight of the composition, including all ranges subsumed therein.

In one aspect, a weight ratio of the compound of formula (I) as described herein, and/or salts or derivatives thereof, and the substituted resorcinol is about 1:40 to 40:1, or about 1:30 to 30:1, or about 1:25 to about 25:1, or about 4:1 to about 1:25, or about 1:10 to 10:1, or about 1:10 to 1:1, or about 10:1 to 1:1, or about 1:8 to about 8:1, or about 1:5 to 5:1, or about 1:5 to 1:1, or about 5:1 to 1:1, or about 1:4 to about 4:1, or about 1:3 to 3:1, or about 1:2 to 2:1, or about 1:2 to 1:1, or about 2:1 to 1:1.

Color stability may be determined through the use of LabScan XE equipment (Hunter Associates Laboratory, Inc. Reston, Virginia). For example. LabScan XE equipment measures color data. Samples at different storage stages housed in vials or in measurement cells are loaded onto the LabScan XE measurement port. By following the measurement procedure specified in the associated instrument menu and computer software, colors (a*, b* and L*) are measured and calculated to result in a color change data (ΔE) value. For reference, a ΔE of approximately three is the threshold where one could visibly detect a color change from the original sample. Alternately, the color of each sample can be directly converted into L*a*b* color scale by uploading pictures of the samples and using standard publicly available software (see, e.g., https://www.ginifab.com or http://colormine.org/convert/rgb-to-hunterlab).

The L* parameter measures darkness and lightness and ranges from black (L*=0) to white (L*=100). The a* parameter measures color content and intensity ranging from green (a*<0) to neutral grey (a*=0) to red (a*>0). The b* parameter also measures color content and intensity ranging from blue (b*<0) to neutral grey (b*=0) to yellow (b*>0). The term ΔE measures the color difference for a given sample at two (2) different time points and is calculated as ΔE=[(L₂*−L₁*)²+(a₂*−a₁*)²+(b₂*−b₁*)²]^{1/2} where L₂*, a₂* and b₂* correspond to the color parameters of the sample at time point 2 (e.g., 12 days) and L₁*, a₁* and b₁* correspond to the color parameters of the sample at time point 1 (e.g., time zero).

In one aspect, the cosmetic compositions as described herein, are color stable. Color stable, as used herein, describes a cosmetic composition that possesses a ΔE of less than 20, preferably less than or equal to 10, on L*, a* and b* at a temperature of about 17° C. to about 25° C. In one aspect, the cosmetic compositions remain color stable for at least 10 days, or at least 12 days, or at least 15 days, or at least 30 days, or at least 45 days, or at least 60 days, or at least 90 days, or at least 120 days, or at least 150 days, up to 365 days. In one aspect, the cosmetic compositions remain color stable for about 1 day to about 10 days, or about 1 day to about 12 days, or about 1 day to about 15 days, or about 1 day to about 30 days, or about 1 day to about 45 days, or about 1 day to about 60 days, or about 1 day to about 90 days, or about 1 day to about 120 days, or about 1 day to about 150 days, or about 1 day to about 365 days. Generally, ΔE≤10 is acceptable, ΔE between 10 to 20 is undesirable, and ΔE>20 is unacceptable.

The “cosmetic composition”, “cosmetically acceptable composition” or “dermatologically acceptable composition”, means that the compositions or components thereof so-described are suitable for use in contact with skin, particularly human skin, without undue toxicity, incompatibility, instability, irritation, or allergic response. Such compositions may be a liquid, cream, pump or aerosol spray, serum, lotion, balm, deodorant, foam, scrub, wash, concentrates and/or powder which can be easily hydrated or applied with an implement or via a face mask or a pad composition. Cosmetic compositions for use in the invention may also be formulated in solid forms such as gels or sticks. In one aspect, combinations of any of the above-described product forms may also be used. the composition is a leave-on composition. The term “leave-on”, as described herein, refers to a composition that is applied to or rubbed on the skin and left thereon.

In addition to the compound of formula (I) as described herein, and/or any salt or derivative thereof, and a substituted resorcinol, the cosmetic composition as described herein, includes a cosmetically acceptable carrier. As used herein, the term “cosmetically acceptable carrier”, “cosmetically acceptable excipient”, “dermatologically acceptable carrier” or “dermatologically acceptable excipient” includes all solvents, dispersion media, coatings, antibacterial and antifungal agents, isotonic and absorption delaying agents and the like, that are cosmetically acceptable or dermatologically acceptable. Except insofar as any conventional media or agent is incompatible with the active ingredient, its use in the cosmetic compositions is contemplated. Supplementary active ingredients can also be incorporated into the compositions. Cosmetically acceptable carriers are suitable for topical application to the skin, have good aesthetic properties, are compatible with the active agents described herein and any other components, and cause minimal or no safety or toxicity concerns. In one aspect, the cosmetically acceptable carrier may comprise an aqueous phase, an oil phase, an alcohol, a silicone phase or a mixture thereof, and may be in the form of an emulsion. Emulsions can have a range of consistencies including thin lotions (which may also be suitable for spray or aerosol delivery), creamy lotions, light creams and heavy creams.

Exemplary emulsions include water-in-oil emulsions, oil-in-water emulsions, silicone-in-water emulsions, water-in-silicone emulsions, polyol-in-silicone emulsions, silicone-in-polyol emulsions, polyol-in-oil emulsions, oil-in-polyol emulsions, wax-in-water emulsions and water-oil-water triple emulsions. Preferred emulsions include oil-in-water (“O/W”) emulsions and water-in-oil (“W/O”) emulsions.

Cosmetic compositions in the form of an emulsion, and suitable for use in the invention, typically have an oil phase containing at one or more cosmetically acceptable fatty materials which may be liquid or solid at room temperature (25° C.).

Suitable cosmetically acceptable fatty materials include naturally derived oils (such as sunflower oil, borage oil, soybean oil, castor oil, olive oil and almond oil); esters of monoalcohols or of polyols with monocarboxylic or polycarboxylic acids, at least one of the alcohols and/or acids comprising at least one hydrocarbon-based chain containing at least 6 carbon atoms (such as octyl palmitate, isopropyl myristate, isopropyl palmitate, isopropyl isostearate, hexyl laurate, isohexyl laurate, isohexyl palmitate, decyl oleate, isodecyl oleate, hexadecyl stearate, decyl stearate, dihexyldecyl adipate, lauryl lactate, myristyl lactate, cetyl lactate, oleyl stearate, oleyl oleate, oleyl myristate, lauryl acetate, cetyl propionate, isononyl isononanoate, propylene glycol dicaprate, diisopropyl adipate, dibutyl adipate, and oleyl adipate); ethers (such as dicapryl ether); fatty alcohols (such as cetyl alcohol, stearyl alcohol and behenyl alcohol); isoparaffins (such as isooctane, isododecane and isohexadecane); silicone oils (such as dimethicones, cyclic silicones, and polysiloxanes); and hydrocarbon oils (such as mineral oil, petrolatum and polyisobutene); fatty acids containing from 8 to 30 carbon atoms (such as stearic acid, lauric acid, palmitic acid and oleic acid); vegetable fats (such as cocoa butter, coconut oil, palm oil and shea butter); natural or synthetic waxes (such as lanolin wax, beeswax, carnauba wax, candelilla wax, paraffin wax, lignite wax, microcrystalline waxes, ceresin, ozokerite, and polyethylene waxes), hydrogenated oils which are solid at 25° C. (such as hydrogenated castor oil, hydrogenated jojoba oil, hydrogenated palm oil, hydrogenated tallow and hydrogenated coconut oil) and fatty esters that are solid at 25° C. (such as C20-40 alkyl stearate). In one aspect, mixtures and/or combinations of any of the above-described materials may also be used. In one aspect, suitable fatty acid materials may be included in the cosmetic composition, as described herein, in an amount may range from about 0.01% to about 20%, or about 0.01% to about 10%, or about 0.01% to 5%, or about 0.1% to about 3%, or about 1% to about 10%, or about 1% to about 8%, by weight of the composition, including all ranges subsumed therein.

Cosmetic compositions in the form of an emulsion, and suitable for use in the invention, typically have an aqueous phase, which may also include one or more organic liquids that are miscible with water at room temperature (25° C.). Exemplary water-miscible organic liquids include monohydric and polyhydric alcohols and derivatives thereof such as C2-C6 alkanols (such as ethanol and isopropanol); C2-C10 glycols and polyols (such as glycerol, propylene glycol, butylene glycol, pentylene glycol, hexylene glycol, caprylyl glycol, dipropylene glycol, and diethylene glycol); C3-C16 glycol ethers (such as mono-, di-, or tripropylene glycol (C1-C4) alkyl ethers and mono-, di-, or triethylene glycol (C1-C4) alkyl ethers) and polyethylene glycol having 2 to 12 oxyethylene units. In one aspect, mixtures and/or combinations of any of the above-described materials may also be used.

Cosmetic compositions in the form of an emulsion, and suitable for use in the invention, generally include surface active ingredients, such as emulsifiers and solubilizers, to enable two or more immiscible components to be combined homogeneously and to help stabilize the composition. In an embodiment, the emulsifier may be selected from the group consisting of anionic, nonionic, cationic and amphoteric surfactants. Emulsifiers that may be used to form O/W or W/O emulsions include sorbitan oleate, sorbitan sesquioleate, sorbitan isostearate, sorbitan trioleate, PEG-20 sorbitan isostearate, polyglyceryl-3-diisostearate, polyglycerol esters of oleic/isostearic acid, polyglyceryl-6 hexaricinolate, polyglyceryl-4-oleate, polyglyceryl-4 oleate/PEG-8 propylene glycol cocoate, polyglyceryl-2 dipolyhydroxystearate, PEG-30 dipolyhydroxystearate, oleamide DEA, TEA myristate, TEA stearate, magnesium stearate, sodium stearate, potassium laurate, potassium ricinoleate, sodium cocoate, sodium tallowate, potassium castorate, sodium oleate, cetyl phosphate, diethanolamine cetyl phosphate, potassium cetyl phosphate, sodium glyceryl oleate phosphate, alkyl sulfates and sulfonates, alkylbenzene sulfonates, alkyl and dialkyl sulfosuccinates, C8-C20 acyl isethionates, C8-C20 acyl methyl isethionates, C8-C20 acyl methyl taurates, dimethicone copolyol, cetyl dimethicone copolyol, octyldimethicone ethoxyglucoside copolyol, dimethicone copolyol crosspolymer, laurylmethicone copolyol, cocoamidopropyl betaine, C12-C20 trialkyl betaines, sodium lauroamphoacetate, and sodium laurodiamphoacetate. In one aspect, mixtures and/or combinations of any of the above-described materials may also be used. In one aspect, surface active ingredient(s) may be included in the cosmetic composition, as described herein, in an amount may range of about 0.1% to about 20%, or about 1% to about 10%, or about 1% to about 8%, by weight of the composition, including all ranges subsumed therein.

Cosmetic compositions for use in the invention may also be formulated in a single-phase carrier such as water and/or one or more water miscible organic liquids (such as the monohydric and polyhydric alcohols and derivatives thereof described above).

Now, it has been surprisingly determined that cosmetic compositions comprising compound of formula (I) as described herein, and a substituted resorcinol, as described herein, are surprisingly and unexpectedly color stable.

### Skin Care Actives

In addition to a compound of general formula (I), a topical composition for use in the invention may include additional skin care actives which may be used to obtain a skin care benefit (e.g., for improving the physical and/or aesthetic characteristics of the skin). Examples of additional skin care actives which are suitable for use in the invention include vitamins, minerals and/or antioxidants, emollients, humectants, skin lightening agents, sunscreens, anti-irritants, exfoliating agents and/or any combinations or mixtures thereof. In one aspect, the cosmetic composition substantially free of sulfates, preferably having less than 5%, or less than 3%, or less than 1%, or less than 0.5%, or less than 0.1% of sulfates, by weight of the composition.

Vitamins, minerals and/or antioxidants suitable for use in the invention include natural botanical antioxidants derived from plant materials such as fruits, vegetables, herbs and spices (such as goji berry, white tea, rosemary, green tea, grape seed and lemongrass extracts); vitamin A and its precursors or derivatives including retinoids such as retinol, beta-carotene, retinal, retinoic acid and C2-20 esters of retinol (such as retinyl palmitate, retinyl propionate, retinyl formate, retinyl acetate, retinyl butyrate, retinyl valerate, retinyl isovalerate, retinyl hexanoate, retinyl heptanoate, retinyl octanoate, retinyl nonanoate, retinyl decanoate, retinyl undecandate, retinyl taurate, retinyl tridecanoate, retinyl myristate, retinyl pentadecanoate, retinyl heptadeconoate, retinyl stearate, retinyl isostearate, retinyl nonadecanoate, retinyl arachidonate, retinyl behenate, retinyl linoleate, and retinyl oleate); vitamin B3 and its precursors or derivatives (such as niacinamide); vitamin B5 and its precursors or derivatives (such as panthenol and its precursors or derivatives); vitamin C and its precursors or derivatives (such as tetrahexyldecyl ascorbate, ascorbyl palmitate); vitamin D and its precursors or derivatives, vitamin E and its precursors or derivatives (such as d-alpha-tocopherol, tocopheryl acetate); vitamin K and its precursors or derivatives; selenium and its derivatives (such as L-selenomethionine); and alpha lipoic acid. In one aspect, mixtures and/or combinations of any of the above-described materials may also be used. In one aspect, vitamins, minerals and/or antioxidants may be included in the cosmetic composition, as described herein, in an amount from about 0.001 to about 10%, or about 0.01% to about 10%, or about 0.01% to about 5%, or about 0.01% to about 4%, or about 0.01% to about 3%, or about 0.1% to about 5%, or about 0.1% to about 4%, or about 0.1% to about 3%, or about 0.5% to about 5%, or about 0.5% to about 4%, or about 0.5% to about 3% by weight of the topical composition, including all ranges subsumed therein.

Emollients suitable for use in the invention include diesters having at least one C8-22 fatty chain (such as dibutyl adipate, diethyl sebacate, diisopropyl dimerate, and dioctyl succinate); branched chain esters having at least one C8-22 fatty chain (such as 2-ethylhexyl myristate, isopropyl stearate and isostearyl palmitate); tribasic acid esters having at least one C8-22 fatty chain (such as triisopropyl trilinoleate and trilauryl citrate), straight chain esters having at least one C8-22 fatty chain (such as lauryl palmitate, myristyl lactate, and stearyl oleate); C10-20 fatty alcohols and acids (such as cetyl, myristyl, palmitic and stearyl alcohols and acids); and C10-40 hydrocarbons (such as mineral oil, petroleum jelly, squalene and isoparaffins). In one aspect, mixtures and/or combinations of any of the above-described materials may also be used. In one aspect, emollient(s) may be included in the cosmetic composition, as described herein, in an amount of about 0.05% to about 6%, or about 0.07% to about 5%, or about 0.1% to about 3%, by weight of the composition, including all ranges subsumed therein.

Humectants suitable for use in the invention include amino acids, chondroitin sulfate, glycerin, diglycerin, triglycerin, polyglycerin, polypropylene glycol, polyethylene glycol, ethylene glycol, diethylene glycol, triethylene glycol, propylene glycol, dipropylene glycol, hexylene glycol, 1,3-butylene glycol, 1,4-butylene glycol, ethylene glycol monoalkyl ether, diethylene glycol monoalkyl ether, erythritol, fructose, glucose, maltose, sucrose, lactose, xylose, inositol, lactitol, xylitol, sorbitol, mannitol, maltitol, panthenol, pentaerythritol, 1,2,6-hexanetriol, honey, hyaluronic acid, hydrogenated honey, hydrogenated starch hydrolysate, natural moisturization factor, PEG-15 butanediol, polyglyceryl sorbitol, sodium and potassium salts of pyrollidone carboxylic acid, sodium glucuronate, trehalose and urea. In one aspect, mixtures and/or combinations of any of the above-described materials may also be used. In one aspect, humectant(s) may be included in the cosmetic composition, as described herein, in an amount of about 0.1% to about 25%, or about 0.5% to about 20%, or about 1% to about 15%, by weight of the topical composition, including all ranges subsumed therein.

Additional skin care actives suitable for use in the invention include natural botanical skin lightening agents derived from plant materials (such as Arctostaphylos patula and Arctostaphylos viscida extracts, Emblica officinalis extract, Mitracarpus scaber extract, Uva ursi (bearberry) extract, Morus bombycis (mulberry) extract, Morus alba (white mulberry) extract, Broussonetia papyrifera (paper mulberry) extract, licorice extract, acerola cherry extract, Chlorella vulgaris extract, Aloe ferrox extract and Rumex occidentalis extract); synthetic or natural sugar amines (such as glucosamine, N-acetyl glucosamine, glucosamine sulfate, mannosamine, N-acetyl mannosamine, galactosamine, N-acetyl galactosamine and their hydrochloride salts); kojic acid and C2-20 esters thereof (such as kojic acid monobutyrate, kojic acid monocaprate, kojic acid monopalmitate, kojic acid monostearate and kojic acid monobenzoate); hydroquinone, hydroquinone monomethyl ether, hydroquinone monoethyl ether, hydroquinone monobenzyl ether, α and β-arbutin, deoxyarbutin (4-[(tetrahydro-2H-pyran-2-yl)oxy]phenol), mequinol (4-hydroxyanisole), glutathione, cysteine, N-acetyl-L-cysteine, azelaic acid, magnesium ascorbyl phosphate, sodium ascorbyl phosphate, peroxides (such as hydrogen peroxide, zinc peroxide, sodium peroxide and benzoyl peroxide); 3-aminotyrosine, glycyrrhizinic acid, petroselinic acid, conjugated linoleic acid and 12- and/or 10-hydroxystearic acid. In one aspect, mixtures and/or combinations of any of the above-described materials may also be used. In one aspect, skin benefit agent(s) may be included in the cosmetic composition, as described herein, in an amount of about 0.001% to about 12%, or about 0.001% to about 10%, or about 0.01% to about 8%, by weight of the composition, including all ranges subsumed therein.

Sunscreens suitable for use in the invention protect the skin from ultraviolet (UV) solar radiation falling within both the UVB region (between 290 nm to 320 nm wavelengths) and the UVA region (between 320 nm and 400 nm wavelengths). Examples of sunscreens include methoxycinnamate derivatives (such as octyl methoxycinnamate and isoamyl methoxycinnamate); camphor derivatives (such as 4-methyl benzylidene camphor, camphor benzalkonium methosulfate, and terephthalylidene dicamphor sulfonic acid); salicylate derivatives (such as octyl salicylate and homosalate); sulfonic acid derivatives (such as phenylbenzimidazole sulfonic acid); benzone derivatives (such as dioxybenzone, sulisobenzone, and oxybenzone); benzoic acid derivatives (such as aminobenzoic acid and octyldimethyl para-amino benzoic acid); octocrylene, diethylhexyl butamido triazone, octyl triazone, butyl methoxydibenzoyl methane, drometrizole trisiloxane, menthyl anthranilate and inorganic UV-absorbing particles (such as zinc oxide and titanium dioxide). In one aspect, mixtures and/or combinations of any of the above-described materials may also be used. In one aspect, sunscreen(s) may be included in the cosmetic composition, as described herein, in an amount of about 0.1% to about 20%, or about 0.5% to about 15%, or about 0.75% to about 10%, by weight of the composition, including all ranges subsumed therein.

Anti-irritants suitable for use in the invention include allantoin, Aloe vera, α-bisabolol, caffeine, chamomile extract, Cola nitada extract, cucumber extract, dipotassium glycyrrhizinate, glycyrrhizic acid, green tea extract, lecithin or hydrogenated lecithin, licorice extract, tea tree oil, strontium acetate, strontium chloride, strontium nitrate, fatty acids with anti-irritant properties (such as linoleic acid and linolenic acid) and aromatic aldehydes with anti-irritant properties (such as 4-methoxy benzaldehyde, 4-ethoxy benzaldehyde, 4-butoxy benzaldehyde and 4-pentoxy benzaldehyde). In one aspect, mixtures and/or combinations of any of the above-described materials may also be used. In one aspect, anti-irritants may be included in the cosmetic composition, as described herein, in an amount of about 0.1% to about 20%, or about 0.5% to about 15%, or about 0.75% to about 10%, by weight of the composition, including all ranges subsumed therein.

Exfoliating agents suitable for use in the invention include benzoyl peroxide, benzoic acid, 3-hydroxy benzoic acid, salicylic acid, acetylsalicylic acid, acetic acid, trichloroacetic acid, 1-pyrrolidone-5-carboxylic acid, α-hydroxy acids (such as glycolic acid, lactic acid, malic acid, tartaric acid, and citric acid); β-hydroxy acids (such as β-hydroxybutyric acid); α-keto acids (such as pyruvic acid, 2-oxopropanoic acid, 2-oxobutanoic acid and 2-oxopentanoic acid); and oxa acids (such as 3,6,9-trioxaundecanedioic acid). In one aspect, mixtures and/or combinations of any of the above-described materials may also be used. In one aspect, exfoliating agent(s) may be included in the cosmetic composition, as described herein, in an amount of about 0.001% to about 12%, or about 0.001% to about 10%, or about 0.01% to about 8%, by weight of the composition, including all ranges subsumed therein.

Another optional skin care active suitable for use includes hemp oil with 2.5 to 25% by weight cannabigerol and/or cannabidiol at from about 0.5% to about 10% by weight. When used, such oil makes up from about 0.0001% to about 12%, or about 0.01% to about 5% by weight of the cosmetic composition, including all ranges subsumed therein.

### Functional Ingredients

In addition, the color stable cosmetic composition as described herein, may include additional functional ingredients.

Examples of additional functional ingredients which are suitable for use in the invention include water soluble or colloidally water soluble polymeric thickening agents (such as hydroxyethyl cellulose, methyl cellulose, hydroxypropyl methyl cellulose, polyquaternium-10, carrageenan, guar gum, hydroxypropyl guar gum, xanthan gum, polyvinylalcohol, acrylic acid/ethyl acrylate copolymers, carboxyvinyl polymers, cross-linked polyacrylate polymers and polyacrylamide polymers); structurant clays (such as magnesium aluminium silicate, attapulgite, bentonite, montmorillonite and hectorite); inorganic pigments (such as titanium oxide, zirconium oxide, cerium oxide zinc oxide, iron oxide, chromium oxide and ferric blue); organic pigments (such as carbon black and barium, strontium, calcium, and aluminium lakes); pearlescent agents (such as mica coated with titanium oxide and/or iron oxide); dyes, preservatives (such as EDTA, disodium EDTA, benzyl alcohol, methylparaben, sodium benzoate, phenoxyethanol, propylparaben, ethylparaben, butylparaben and isobutylparaben); pH adjusters and fragrances. In one aspect, mixtures and/or combinations of any of the above-described materials may also be used. In one aspect, the cosmetic composition is substantially free of parabens, having less than 5%, or less than 3%, or less than 1%, or less than 0.5%, or less than 0.1%, or 0% of parabens. In one aspect, functional ingredient(s) may be included in the cosmetic composition, as described herein, in an amount of about 0.001% to about 12%, or about 0.001% to about 10%, or about 0.01% to about 8%, by weight of the composition, including all ranges subsumed therein.

Many types of packaging can be used to store and deliver the present cosmetic composition. The selection of packaging is dependent upon the personal care end-use and the viscosity of the composition itself. In one aspect, a leave-on product may simply be stored in a non-deformable bottle, post-consumer (PCR) resin or squeeze container, such as a tube or a lidded jar. In one aspect, a leave-on product, such as lotions and creams for skin, typically employ plastic containers with an opening at a dispense end covered by an appropriate closure. Conventional closures include flip-top hinged lids, screw-caps and non-aerosol pumps. As another example, appropriate packaging to be used for antiperspirants, deodorants and depilatories include a container with a roller-ball applicator on a dispensing end if the composition is fluid and of a thinner viscosity. If the composition is in a stick format, a container with a propel-repel mechanism wherein the stick is fixed on a platform towards a dispensing orifice is appropriate. If the composition is in an aerosol format, then metallic cans pressurized by a propellant and having a spray nozzle is appropriate. In general, patches, bottles, tubes, roller-ball applicators, squeeze containers or lidded jars are preferred.

In one aspect, a method is provided for making a color stable cosmetic composition comprising: mixing a compound of formula (I) as described herein, and/or salts or derivatives thereof, and a substituted resorcinol to a cosmetically acceptable base, wherein a weight ratio of the compound of formula (I), and/or salts or derivatives thereof, and the substituted resorcinol is about 1:40 to 40:1, or about 1:10 to 10:1, or about 5:1 to 1:5, or about 3:1 to 1:3, wherein the cosmetic composition is color stable for at least 10 days.

In one aspect, a method is provided for making a color stable cosmetic composition comprising: mixing a compound of formula (I) as described herein, and/or salts or derivatives thereof, and a substituted resorcinol to a cosmetically acceptable base, wherein an amount of the substituted resorcinol, by weight of the composition is about 0.01%-10%, or about 0.01%-5%, or about 0.01%-3%, or about 0.1%-5%, or about 0.1%-3%, or about 0.1%-1%; and wherein an amount of the compound of formula (I), and/or salts and derivatives thereof, by weight of the composition is about 0.01%-10%, or about 0.01%-5%, or about 0.01%-3%, or about 0.1%-5%, or about 0.1%-3%, or about 0.1%-1%, wherein the cosmetic composition is color stable for at least 10 days.

In one aspect, a method is provided for imparting a skin benefit to skin comprising: applying a cosmetic composition to the skin, wherein the cosmetic composition comprises a compound of formula (I) as described herein, and/or salts or derivatives thereof, and a substituted resorcinol to a cosmetically acceptable base, wherein a weight ratio of the compound of formula (I), and/or salts or derivatives thereof, and the substituted resorcinol is about 1:40 to 40:1, or about 1:10 to 10:1, or about 5:1 to 1:5, or about 3:1 to 1:3.

In one aspect, a method is provided for imparting a skin benefit to skin comprising: applying a cosmetic composition to the skin, wherein the cosmetic composition comprises a compound of formula (I) as described herein, and/or salts or derivatives thereof, and a substituted resorcinol to a cosmetically acceptable base, wherein an amount of the substituted resorcinol, by weight of the composition is about 0.01%-10%, or about 0.01%-5%, or about 0.01%-3%, or about 0.1%-5%, or about 0.1%-3%, or about 0.1%-1%; and wherein an amount of the compound of formula (I), and/or salts and derivatives thereof, by weight of the composition is about 0.01%-10%, or about 0.01%-5%, or about 0.01%-3%, or about 0.1%-5%, or about 0.1%-3%, or about 0.1%-1%.

As used herein, “administering to skin in need of such treatment” or “applying to skin in need of such treatment” means contacting (e.g., by use of the hands or an applicator such as, but not limited to, a wipe, tube, roller, spray, or patch) the area of skin in need such treatment or an area of skin proximate to the area of skin in need of such treatment. The cosmetic composition is suitably applied to the skin, preferably facial skin, at the rate of one or two applications per day. Generally, an amount corresponding to about 1 to 2 ml of the composition per application is applied uniformly over the area of treatment twice daily for a period of at least 7 (seven) days, more preferably at least 30 (thirty) days.

The following Examples are provided to further illustrate an understanding of the invention. The Examples are not intended to limit the scope of the claims.

### EXAMPLES

### Examples 1-6: Color Stabilization Effect of Indoles on 4HR-Induced Color Formation in Solution after 12 Days at about 21° C. To 24° C.

Samples for color evaluation were prepared by dissolving 4-hexyl resorcinol (4HR) in acetone:water (86:14) at desired percent level (% wt/wt basis). Indoles, namely, 2-(1H-indol-3-yl)acetic acid (or “|3AA”), 3-(1H-indol-3-yl)propanoic acid (or “13PA”), 4-(1H-indol-3-yl)butanoic acid (or “13BA”), diindolylmethane (“DIM”), and pyroglutamylamidoethyl indole (“PGEI”) were added at the desired percent level (% wt/wt basis) and the solutions kept at between about 21-24° C. for the desired amount of time (i.e., 12 days). The color of each solution was assessed visually and converted into L*a*b* scale using publicly available software and experimentally using a Labscan XE instrument (Hunter Associates Labs Inc., Reston, VA) and processed with Universal Software (version 4.10).

TABLE 1

| | | Day 0 | | | Day 12 | | | |
|---|---|---|---|---|---|---|---|---|
| Ex. | Sample | L* | a* | b* | L* | a* | b* | ΔE |
| 1 | 1% 4HR | 87.5 | 5.6 | 8.2 | 64.3 | 18.9 | 11 | 26.9 |
| 2 | 1% 4HR + 1% | 92.5 | −8.4 | 21.9 | 83.1 | −6.2 | 22 | 9.6 |
| | I3AA | | | | | | | |
| 3 | 1% 4HR + 1% | 87.5 | −4.4 | 21.9 | 78.1 | −3.3 | 21.8 | 9.4 |
| | I3PA | | | | | | | |
| 4 | 1% 4HR + 1% | 89.2 | −6.0 | 22.1 | 80.1 | −5.5 | 22.3 | 9.1 |
| | I3BA | | | | | | | |
| 5 | 1% 4HR + 1% | 89.3 | −5.6 | 32.5 | 48.7 | 22.0 | 23.3 | 49.9 |
| | DIM | | | | | | | |
| 6 | 1% 4HR + 1% | 76.8 | 1.0 | 22.0 | 61.6 | 1.1 | 22.4 | 15.2 |
| | PGEI | | | | | | | |

Table 1 above shows the ability of various indoles (at 1%) to stabilize the color formation induced by 4HR (at 1%) in solution after 12 days. After 12 days, a solution of 4HR generates an unacceptable level of color (dark orange-pink) by visual assessment and as determined by the ΔE value of 26.9. Surprisingly and unexpected, this unacceptable color formation is inhibited by the addition of 1% of 13AA, 13PA or 13BA to generate solutions with visually and experimentally acceptable color (ΔE of 9.6, 9.4 and 9.1, respectively). By contrast, other indoles outside the scope of the invention, i.e., DIM and PGEI, were significantly less effective at preventing color formation induced by 4HR, as each of these indoles had an experimentally unacceptable or undesirable color (ΔE of 49.9 and 15.2, respectively).

### Examples 7-11: Color Stabilization Effect of Indoles on 4ER-Induced Color Formation in Solution after 12 Days at about 21° C. To 24° C.

Samples for color evaluation were prepared by dissolving 4-ethyl resorcinol (4ER) in acetone:water (86:14) at desired percent level (% wt/wt basis). Indoles, namely, 13AA, 13PA, 13BA, and DIM were added at the desired percent level (% wt/wt basis) and the solutions kept at between about 21-24° C. for the desired amount of time (i.e., 12 days). The color of each solution was assessed visually and converted into L*a*b* scale using publicly available software and experimentally using a Labscan XE instrument (Hunter Associates Labs Inc., Reston, VA) and processed with Universal Software (version 4.10).

TABLE 2

| | | Day 0 | | | Day 12 | | | |
|---|---|---|---|---|---|---|---|---|
| Ex. | Sample | L* | a* | b* | L* | a* | b* | ΔE |
| 7 | 1% 4ER | 85.4 | −0.7 | 9.35 | 62.6 | 13.7 | 16.0 | 27.8 |
| 8 | 1% 4ER + 1% | 83.2 | −5.9 | 12.2 | 74.9 | −5.8 | 11.5 | 8.3 |
| | I3AA | | | | | | | |
| 9 | 1% 4ER + 1% | 77.6 | −3.9 | 11.4 | 75.1 | −6.9 | 13.3 | 4.3 |
| | I3PA | | | | | | | |
| 10 | 1% 4ER + 1% | 75.5 | −3.8 | 12.1 | 72.0 | −4.0 | 13.0 | 3.6 |
| | I3BA | | | | | | | |
| 11 | 1% 4ER + 1% | 82.7 | −1.5 | 12.6 | 49.2 | 13.9 | 21.6 | 37.9 |
| | DIM | | | | | | | |

Table 2 above shows the ability of various indoles (at 1%) to stabilize the color formation induced by 4ER (at 1%) in solution after 12 days. After 12 days, a solution of 4ER generates an unacceptable level of color (dark pink) by visual assessment and as determined by the ΔE value of 27.8. Surprisingly and unexpected, this unacceptable color formation is inhibited by the addition of 1% of 13AA, 13PA or 13BA to generate solutions with visually and experimentally acceptable color (ΔE of 8.3, 4.3, and 3.6, respectively). By contrast, other indoles outside the scope of the invention, i.e., DIM, were significantly less effective at preventing color formation induced by 4ER, this indole had an experimentally unacceptable color (ΔE of 37.9).

### Examples 12-16: Color Stabilization Effect of Indoles on Isobutylamido Thiazolyl Resorcinol (ITR)-Induced Color Formation in Solution after 12 Days at about 21° C. To 24° C.

Samples for color evaluation were prepared by dissolving isobutylamido thiazolyl resorcinol (ITR) in acetone:water (86:14) at desired percent level (% wt/wt basis). Indoles, namely, 13AA, 13PA, 13BA, and DIM were added at the desired percent level (% wt/wt basis) and the solutions kept at between about 21-24° C. for the desired amount of time (i.e., 12 days). The color of each solution was assessed visually and converted into L*a*b* scale using publicly available software and experimentally using a Labscan XE instrument (Hunter Associates Labs Inc., Reston, VA) and processed with Universal Software (version 4.10).

TABLE 3

| | | Day 0 | | | Day 12 | | | |
|---|---|---|---|---|---|---|---|---|
| Ex. | Sample | L* | a* | b* | L* | a* | b* | ΔE |
| 12 | 1% ITR | 84.1 | −1.7 | 13.9 | 78.7 | −6.4 | 38.4 | 26.5 |
| 13 | 1% ITR + 1% | 79.4 | −5.4 | 14.2 | 74.4 | −6.0 | 22.0 | 9.4 |
| | I3AA | | | | | | | |
| 14 | 1% ITR + 1% | 76.2 | −4.5 | 12.9 | 75.0 | −6.8 | 23.0 | 10.5 |
| | I3PA | | | | | | | |
| 15 | 1% ITR + 1% | 75.2 | −3.6 | 14.7 | 64.7 | −2.1 | 18.2 | 11.2 |
| | I3BA | | | | | | | |
| 16 | 1% ITR + 1% | 86.1 | −2.3 | 16.6 | 60.9 | 14.1 | 33.3 | 34.4 |
| | DIM | | | | | | | |

Table 3 above shows the ability of various indoles (at 1%) to stabilize the color formation induced by ITR (at 1%) in solution after 12 days. After 12 days, a solution of ITR generates an unacceptable level of color (dark yellow) by visual assessment and as determined by the ΔE value of 26.5. Surprisingly and unexpected, this unacceptable color formation is inhibited by the addition of 1% of I3AA, I3PA or I3BA to generate solutions with visually and experimentally acceptable color (ΔE 9.4 for I3AA) or undesirable color (ΔE 10.5 and 11.2 for I3PA and I3BA, respectively). By contrast, other indoles outside the scope of the invention, i.e., DIM, were significantly less effective at preventing color formation induced by ITR, this indole had an experimentally unacceptable color (ΔE of 34.4).

### Examples 17-21: Color Stabilization Effect of Different Levels of I3AA on 4HR-Induced Color Formation in Solution after 12 Days at about 18° C. To 21° C.

Samples for color evaluation were prepared by dissolving 4HR in acetone:water (86:14) at desired percent level (% wt/wt basis). An indole, namely, I3AA, was added at the desired percent level (% wt/wt basis) and the solutions kept at between about 18-21° C. for the desired amount of time (i.e., 12 days). The color of each solution was assessed visually and converted into L*a*b* scale using publicly available software and experimentally using a Labscan XE instrument (Hunter Associates Labs Inc., Reston, VA) and processed with Universal Software (version 4.10).

TABLE 4

| | | Day 0 | | | Day 12 | | | |
|---|---|---|---|---|---|---|---|---|
| Ex. | Sample | L* | a* | b* | L* | a* | b* | ΔE |
| 17 | 1% 4HR | 91.4 | −0.5 | 16.8 | 71.5 | 5.26 | 19.6 | 20.9 |
| 18 | 1% 4HR + 0.1% | 93.8 | −9.1 | 25.8 | 79.3 | −4.8 | 18.5 | 16.8 |
| | I3AA | | | | | | | |
| 19 | 1% 4HR + 0.5% | 94.3 | −9.3 | 26.2 | 82.4 | −8.6 | 23.2 | 12.2 |
| | I3AA | | | | | | | |
| 20 | 1% 4HR + 5% | 86.6 | −8.7 | 30.4 | 78.1 | −5.8 | 28.3 | 9.2 |
| | I3AA | | | | | | | |
| 21 | 1% 4HR + 10% | 90.9 | −10 | 38.1 | 78.1 | −2.4 | 39 | 14.9 |
| | I3AA | | | | | | | |

Table 4 above shows the ability of various levels of indole 13AA to stabilize the color formation induced by 4HR (at 1%) in solution after 12 days. After 12 days, a solution of 4HR generates an unacceptable level of color (dark orange-pink) by visual assessment and as determined by the ΔE value of 20.9. Surprisingly and unexpected, this unacceptable color formation is inhibited by the addition of 13AA at 5% level to generate a solution with visually and experimentally acceptable color (ΔE of 9.2). Improvements in color, albeit in the undesirable color range, were also observed with 0.1%, 0.5%, and 10% 13AA.

### Examples 22-26: Color Stabilization Effect of Indoles on Combined 4HR+B3-Induced Color Formation in Solution after 15 Days at about 21° C. To 24° C.

Samples for color evaluation were prepared by dissolving 4HR and niacinamide (B3) in acetone:water (86:14) at desired percent level (% wt/wt basis). Indoles, namely, 13AA, 13PA, 13BA, and DIM were added at the desired percent level (% wt/wt basis) and the solutions kept at between about 21-24° C. for the desired amount of time (i.e., 15 days). The color of each solution was assessed visually and converted into L*a*b* scale using publicly available software and experimentally using a Labscan XE instrument (Hunter Associates Labs Inc., Reston, VA) and processed with Universal Software (version 4.10).

TABLE 5

| | | Day 0 | | | Day 15 | | | |
|---|---|---|---|---|---|---|---|---|
| Ex. | Sample | L* | a* | b* | L* | a* | b* | ΔE |
| 22 | 1% 4HR + 1% | 81.6 | 1.65 | 20.1 | 56.8 | 14.4 | 18.9 | 28 |
| | B3 | | | | | | | |
| 23 | 1% 4HR + 1% | 75.4 | −6.9 | 22.3 | 70.7 | −6.6 | 18.9 | 5.9 |
| | B3 + 1% I3AA | | | | | | | |
| 24 | 1% 4HR + 1% | 75.3 | −5.9 | 22.6 | 69.7 | −5.8 | 20.7 | 5.9 |
| | B3 + 1% I3PA | | | | | | | |
| 25 | 1% 4HR + 1% | 73.7 | −4.1 | 24.1 | 70.9 | −4.5 | 20.2 | 4.9 |
| | B3 + 1% I3BA | | | | | | | |
| 26 | 1% 4HR + 1% | 70.3 | 2.53 | 21.5 | 51.6 | 11.4 | 21.4 | 20.8 |
| | B3 + 1% DIM | | | | | | | |

Table 5 above shows the ability of various indoles (at 1%) to stabilize the color formation induced by a combination of 4HR+B3 (both at 1%) in solution after 15 days. After 15 days, a solution of 4HR+B3 generates an unacceptable level of color (dark orange-pink) by visual assessment and as determined by the ΔE value of 28. Surprisingly and unexpected, this unacceptable color formation is inhibited by the addition of 1% of 13AA, 13PA or 13BA to generate solutions with visually and experimentally acceptable color (ΔE of 5.9, 5.9 and 4.9, respectively). By contrast, other indoles outside the scope of the invention, i.e., DIM, were significantly less effective at preventing color formation induced by 4HR+B3, this indole had an experimentally unacceptable color (ΔE of 20.8).

### Examples 27-33: Color Stabilization Effect of Indoles on Combined 4HR+B3-Induced Color Formation in Solution after 10 Days at about 21° C. To 24° C.

Samples for color evaluation were prepared by dissolving 4HR and niacinamide (B3) in acetone:water (86:14) at desired percent level (% wt/wt basis). Indoles, namely, 13AA, 13PA, and DIM were added at the desired percent level (% wt/wt basis) and the solutions kept at between about 21-24° C. for the desired amount of time (i.e., 10 days). The color of each solution was assessed visually and converted into L*a*b* scale using publicly available software and experimentally using a Labscan XE instrument (Hunter Associates Labs Inc., Reston, VA) and processed with Universal Software (version 4.10).

TABLE 6

| | | Day 0 | | | Day 10 | | | |
|---|---|---|---|---|---|---|---|---|
| Ex. | Sample | L* | a* | b* | L* | a* | b* | ΔE |
| 27 | 0.4% 4HR + 3% | 93.3 | −2.6 | 10.8 | 72.2 | 1.78 | 8.6 | 21.7 |
| | B3 | | | | | | | |
| 28 | 0.4% 4HR + 3% | 86.3 | −10.0 | 23.0 | 79.5 | −6.1 | 9.1 | 16.1 |
| | B3 + 0.1% I3AA | | | | | | | |
| 29 | 0.4% 4HR + 3% | 86.9 | −6.9 | 13.6 | 81.7 | −6.3 | 11.5 | 5.6 |
| | B3 + 1% I3AA | | | | | | | |
| 30 | 0.4% 4HR + 3% | 94.2 | −8.9 | 21.1 | 89.8 | −9.7 | 30.7 | 10.6 |
| | B3 + 5% I3AA | | | | | | | |
| 31 | 0.4% 4HR + 3% | 90.7 | −7.5 | 25.8 | 82.3 | −5.8 | 35.6 | 13.1 |
| | B3 + 10% I3AA | | | | | | | |
| 32 | 0.4% 4HR + 3% | 89.7 | −6.6 | 15.3 | 82.9 | −7.0 | 13.2 | 7.2 |
| | B3 + 1% I3PA | | | | | | | |
| 33 | 0.4% 4HR + 3% | 97.3 | −6.4 | 13.3 | 74.9 | −1.7 | 15.6 | 23.0 |
| | B3 + 1% DIM | | | | | | | |

Table 6 above shows the ability of various indoles to stabilize the color formation induced by a combination of 4HR+B3 (at 0.4% and 3%, respectively) in solution after 10 days. After 10 days, a solution of 4HR+B3 generates an unacceptable level of color (dark orange-pink) by visual assessment and as determined by the ΔE value of 21.7.

Surprisingly and unexpected, this unacceptable color formation is inhibited by the addition of 13AA at 1% level to generate a solution with visually and experimentally acceptable color (ΔE of 5.6). Improvements in color, albeit in the undesirable color range, were also observed with 5%, 10% and 0.1% levels of 13AA (ΔE of 10.6, 13.1 and 16.1, respectively). Also surprisingly, other indoles within the scope of the present application were able to generate solutions with visually and experimentally acceptable color (ΔE of 7.2, with 1% 13PA).

By contrast, other indoles outside the scope of the invention, i.e., DIM, were significantly less effective at preventing color formation induced by 4HR+B3, this indole had an experimentally unacceptable color (ΔE of 23).

1.-11. (canceled)

12. A cosmetic composition comprising:
a compound of formula (I): 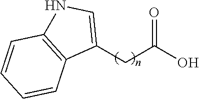
or any salts or derivatives thereof; and
a substituted resorcinol selected from 4-ethylresorcinol, 4-hexylresorcinol, isobutylamido thiazolyl resorcinol, or any combination thereof,
wherein n is 1 to 3;
wherein a weight ratio of the compound of formula (I) and the substituted resorcinol is from 1:40 to 40:1.

13. The cosmetic composition of claim 12, wherein the weight ratio of the compound of formula (I) and the substituted resorcinol is from 1:10 to 10:1.

14. The cosmetic composition of claim 12, wherein the weight ratio of the compound of formula (I) and the substituted resorcinol is from 5:1 to 1:5.

15. The cosmetic composition of claim 12, wherein the weight ratio of the compound of formula (I) and the substituted resorcinol is from 3:1 to 1:3.

16. The cosmetic composition of claim 12, wherein the composition is color stable such that it possesses a ΔE of less than 20, on L*, a* and b* at a temperature of about 17° C. to about 25° C.

17. The cosmetic composition of claim 12, wherein the compound of formula (I) is 2-(1H-indol-3-yl)acetic acid, 3-(1H-indol-3-yl)propanoic acid, 4-(1H-indol-3-yl)butanoic acid, or any salts or derivatives thereof.

18. The cosmetic composition of claim 12, wherein the composition comprises from 0.01% to 10%, by weight of the compound of formula (I).

19. The cosmetic composition of claim 12, wherein the composition comprises from 0.01% to 10%, by weight of the substituted resorcinol.

20. The cosmetic composition of claim 12 further comprising at least one vitamin selected from vitamin A and its precursors or derivatives, vitamin B3, vitamin C, vitamin D, vitamin E, vitamin K, or any combinations thereof.

21. The cosmetic composition of claim 20, wherein the at least one vitamin is vitamin B3.

22. The cosmetic composition of claim 12 further comprising at least one antioxidant, emollient, humectant, skin lightening agent, sunscreen active, anti-irritant, exfoliating agent, or any combinations thereof.

23. A cosmetic composition comprising:
from 0.01 to 3%, by weight of 2-(1H-indol-3-yl)acetic acid, 3-(1H-indol-3-yl)propanoic acid, 4-(1H-indol-3-yl)butanoic acid, or any salts or derivatives thereof; and
from 0.01 to 3%, by weight of a substituted resorcinol selected from 4-ethylresorcinol, 4-hexylresorcinol, isobutylamido thiazolyl resorcinol, or any combination thereof,
wherein the composition is stable.

24. The cosmetic composition of claim 23 further comprising at least one vitamin selected from vitamin A and its precursors or derivatives, vitamin B3, vitamin C, vitamin D, vitamin E, vitamin K, or any combinations thereof.

25. The cosmetic composition of claim 24, wherein the at least one vitamin is vitamin B3.

26. The cosmetic composition of claim 23 further comprising at least one antioxidant, emollient, humectant, skin lightening agent, sunscreen active, anti-irritant, exfoliating agent, or any combinations thereof.

27. A method of providing a skin benefit to skin comprising applying a color stable cosmetic composition to skin, wherein the cosmetic composition comprises a compound of formula (I): 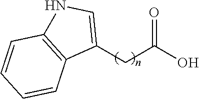
or any salts or derivatives thereof, wherein n is 1 to 3; and
a substituted resorcinol to a cosmetically acceptable base,
wherein a weight ratio of the compound of formula (I) and the substituted resorcinol is from about 1:10 to 10:1.

28. The method of claim 27, wherein the substituted resorcinol is selected from 4-ethylresorcinol, 4-hexylresorcinol, isobutylamido thiazolyl resorcinol, or any combination thereof.

29. The method of claim 27, wherein the compound of formula (I) is 2-(1H-indol-3-yl)acetic acid, 3-(1H-indol-3-yl)propanoic acid, 4-(1H-indol-3-yl)butanoic acid, or any salts or derivatives thereof.
